(19) United States
(12) Patent Application Publication
(10) Pub. No.: US 2024/0076248 A1
(43) Pub. Date: Mar. 7, 2024

(54) METHOD FOR PURIFICATION OF RADIOLABELED MACROAGGREGATED HUMAN SERUM ALBUMIN

(71) Applicant: Trasis S.A., ANS (BE)

(72) Inventors: Thomas VERGOTE, MARILLES (BE); Julien MASSET, NOVILLE SUR MEHAIGNE (BE); Corentin WARNIER, NESSONVAUX (BE); Jean-Luc MORELLE, LIEGE (BE); Charles VRIAMONT, AVIN (BE); Frederique BLANC-BEGUIN, PLOUGASTEL-DAOULAS (FR); Pierre-Yves LEROUX, PLOUGASTEL-DAOULAS (FR); Peter EU, Victoria (AU)

(21) Appl. No.: 18/261,493

(22) PCT Filed: Jan. 24, 2022
(86) PCT No.: PCT/EP2022/051434

§ 371 Date: Jul. 14, 2023

(30) Foreign Application Priority Data
Jan. 28, 2021 (BE) 2021/5067

(51) Int. Cl. C07B 59/00; A61K 51/08; A61K 51/12; C07K 1/34

(52) U.S. Cl. CPC C07B 59/008; A61K 51/081; A61K 51/1255; C07K 1/34; C07B 2200/05

(57) ABSTRACT

A method for synthesis and purification of radiolabeled macroaggregated human serum albumin (MAA) to form a bulk solution injectable to a patient includes: providing a radiometal in a generator, the radiometal being a generator eluate; synthesizing the radiolabeled MAA in a reactor with MAA particles from a commercially available labeling kit for 99mTc and the generator eluate so as to provide synthesized radiolabeled MAA particles; passing the synthesized radiolabeled MAA particles on a syringe filter membrane having a membrane composition, diameter, and pore size for trapping the radiolabeled MAA particles as trapped radiolabeled MAA particles and not retaining impurities from a bulk solution, the impurities including free radioactive metal isotopes, parent radioactive metal breakthrough, and stannous chloride present in the MAA labeling kit for 99mTc; and untrapping the trapped radiolabeled MAA particles from the syringe filter using a saline or buffered solution passing through the syringe filter.

### CROSS-REFERENCE TO PRIOR APPLICATIONS

This application is a U.S. National Phase application under 35 U.S.C. § 371 of International Application No. PCT/EP2022/051434, filed on Jan. 24, 2022, and claims benefit to Belgian Patent Application No. BE 2021/5067, filed on Jan. 28, 2021. The International Application was published in French on Aug. 4, 2022 as WO/2022/161897 under PCT Article 21(2).

### FIELD

The present invention relates to a simplified method for the purification of radiometallabeled macroaggregated human serum albumin (MAA), more simply referred to as radiolabeled herein below. This simplification allows easier automation of such radiotracer syntheses.

### BACKGROUND

Positron Emission Tomography

Positron emission tomography (PET) is a medical imaging method for obtaining quantitative molecular and biochemical information of physiological processes in the body. The most common PET radiopharmaceutical in use today is [18F]-fluorodeoxyglucose ([18F]-FDG), a radiolabeled glucose molecule. PET imaging with [18F]-FDG allows to visualize glucose metabolism and has a broad range of clinical indications. Among positron emitters, 18F is the most widely used today in the clinical environment. Due to the increasing regulatory pressure, the radiopharmaceuticals are usually prepared today on single use components assembled in ready-to-use cassettes.

Besides 18F, radio-metals (e.g. 64Cu, 89Zr, 67Ga, 68Ga, 86Y, 90Y, 177Lu and 99mTc) play a pivotal role in nuclear medicine as therapeutic and imaging agents for radiation therapy and labeling of biologically important low-molecular weight molecules and macromolecules like proteins, peptides and antibodies.

In the recent past, a rapid increase has been noted in both clinical and preclinical studies involving 68Ga-labeled radiopharmaceuticals (Velikyan I., Prospective of 68Ga-radiopharmaceutical development. Theranostics 2014; 4:47-80; Banerjee S. R., Pomper M. G. Clinical applications of Gallium-68. Appl. Radiat. Isot. 2013; 76:2-13; Zimmerman B. E. Current status and future needs for standards of radionuclides used in positron emission tomography. Appl. Radiat. Isot. 2013; 76:31-37; Smith D. L., Breeman W. A. P., Sims-Mourtada J., The untapped potential of Gallium-68 PET: The next wave of 68Ga-agents. Appl. Radiat. Isot. 2013; 76:14-23). This increase can be attributed to the favorable physical characteristics of 68Ga (Eβₘₐₓ 1.8 MeV, β+89%, T_{1/2}=67.7 minutes, against about 6 hours for 99mTc) for imaging various rapidly changing processes (proliferation, apoptosis, angiogenesis) and targets (growth hormones, myocardial and pulmonary perfusion, inflammation and infection), and to some extent, to newer, more reliable production and labeling methods. For example, Gallium-68 labeled somatostatin analogs have already shown their superiority over the existing agent 111In-DTPA-octreotide through enhanced sensitivity, specificity, accuracy and cost effectiveness for the diagnosis of patients with neuroendocrine tumors (Oberg K., Gallium-68 somatostatin receptor PET/CT: Is it time to replace 111Indium DTPA octrotide for patients with neuroendocrine tumors? Endocrine 2012; 42:3-4; Schreiter N. F., Brenner W., Nogami M., Buchert R., Huppertz A., Pape U. F., Prasad V., Hamm B., Maurer M. H., Cost comparison of 111In-DTPA-octrotide scintigraphy and 68Ga-DOTATOC PET/CT for staging enteropancreatic neuroendocrine tumours. Eur. J. Nucl. Med. Mol. Imaging 2012; 39: 72-82; Hofman M. S., Kong G., Neels O. C., Eu P., Hong E., Hicks R. J., High management impact of Ga-68 DOTATATE (GaTate) PET/CT for imaging neuro-endocrine and other somatostatin expressing tumours. J. Med. Imaging Radiat. Oncol. 2012; 56-40-47).

Another reason for the current Gallium-68 infatuation is that it can be produced onsite by widely commercially available 68Ge/68Ga generators. Such 68Ge/68Ga generators are broadly accessible in nuclear medicine facilities not equipped with an on-site cyclotron. The simplicity and lower capital cost of the 68Ge/68Ga generator have made it more popular among the nuclear medicine facilities with relatively lower number of requirements for 68Ga-labeled doses (Rosch F. Past, present and future of 68Ge/68Ga generators. Appl. Radiat. Isot. 2013; 76:24-30).

Radiometallabeled Macroaggregated Human Serum Albumin (MAA)

The use of macroaggregated human serum albumin (MAA) as a perfusion agent has been evaluated since 1965 (Furth E. D., Okinaka A. J., Focht E. F., Becker D. V., The distribution, metabolic fate and radiation dosimetry of 131I labelled macroaggregated albumin. J. Nucl. Med. 1965; 6:506-518). In 1974, an instant kit for the preparation of 99mTc-labeled MAA was evaluated for this purpose using Single Photon Emission Computed Tomography SPECT (Charidra R., Shamoun J., Braunstein P., DuHov O. L., Clinical evaluation of an instant kit for preparation of 99mTc MAA for lung scanning. J. Nucl. Med. 1974; 14-9:702-705). The drug became the standard for lung perfusion studies and still dominates the market (Suga K., Kawakami Y., Zaki M., Yamashita T., Matsumoto T., Matsunaga N., Pulmonary perfusion assessment with respiratory gated Tc-99m macroaggregated albumin SPECT: preliminary results. Nucl. Med. Commun. 2004; 25: 183-193). Nowadays, numerous FDA-approved MAA labeling kits for 99mTc are commercially available (e.g. Pulmocis® (from CisBio), LyoMAA® (from Covidien), HAS-B20 ® (from Rotop), MAASOL® (from GE), etc.). All those kits are provided under the form of a sterile single use vial that contains ˜2.0 mg of MAA particles, ˜0.05 mg of SnCl₂ (as a 99mTc reduction agent) and ˜5.0 mg of free albumin.

Set against the background of the worldwide shortage of 99Mo, which decays to form the 99mTc and is used in about 600,000 medical imaging procedures worldwide every week, it is necessary to think about alternatives in order to be independent of any lack of 99mTc. The 68Ge/68Ga generator represents such an attractive alternative. Furthermore, PET/CT delivers images with significantly higher resolution than SPECT. Thus, 68Ga-labeled MAA for PET/CT perfusion imaging represents an attractive alternative to 99mTc-labeled MAA.

MAA was first successfully labeled with 68Ga in 1986 (Maziere B., Loc'h C., Steinling M., Comar D., Stable labelling of serum albumin microspheres with gallium-68. Int. J. Radiat. Appl. Instrum. Part A 1986; 37:360-361) and in 1989 (Even G. A., Green M. A., Gallium-68-labeled macroaggregated human serum albumin, 68Ga-MAA. Int. J. Radiat. Appl. Instr. 1989; 16:319-321) but never used at that time, probably due to the lack of reliability of the existing 68Ge/68Ga generators and low availability of PET imaging cameras. Later on, Mathias et al. (Mathias C. J., Green M. A., A convenient route to [68Ga]Ga-MAA for use as a particulate PET perfusion tracer. Appl. Radiat. Isot. 2008; 66:1910-1912) successfully labelled MAA with 68Ga as well. Similar results have been reported by using commercially available 99mTc-MAA kit systems (Jain A., Subramanian S., Pandey U., Sarma H. D., Ram R., Dash A., In-house preparation of macroaggregated albumin (MAA) for 68Ga labelling and its comparison with commercially available MAA. J. Radioanal. Nucl. Chem. 2016; 308:817-824; Amor-Coarasa A., Milera A., Carvajal D., Gulec S., McGoron A. J., Lyophilized kit for the preparation of the PET perfusion agent [68Ga]-MAA. Int. J. Mol. Imaging 2014:1-7; Ament S. J., Maus S., Reber H., Buchholz H. G., Bausbacher N., Brochhausen C., Graf F., Miederer M., Schreckenberger M., PET lung ventilation/perfusion imaging using 68Ga aerosol (Galligas) and 68Ga-labeled macroaggregated albumin. Recent Results Cancer Res. 2013; 194:395-423). To remove undesired components like stannous chloride, which is usually used as a reduction component, the lyophilizate of the MAA-kit system was resuspended and washed with 0.9% saline using centrifugation. A pre-conjugation of MAA with the DOTA chelator (Kotzerke J., Andreeff M., Wunderlich G., Wiggermann P., Zphel K., Ventilation/Perfusion scans using Ga-68 labeled tracers. Abstracts of invited lectures. World J. Nucl. Med. 2011; 10:26-59) for an efficient 68Ga labelling is not needed in this procedure. After labeling, the 68Ga-MAA were purified using centrifugation, which is time-consuming, decreases significantly the final yield and is not prone to automation. The authors could also show that there was no difference in morphology between unlabeled and labeled MAA particles. Maus et al. (Maus S., Buccholz H. G., Ament S., Brochhausen C., Bausbacher N., Schreckenberger M., Labelling of commercially available human serum albumin kits with 68Ga as surrogates for 99mTc-MAA microspheres. Appl. Radiat. Isot. 2011; 69:171-175) found similar results and used this method for investigation of the labeling efficiency by using HEPES buffer. A maximum labeling efficiency of 70% has been found and the radiochemical purity after a final Solid Phase Extraction (SPE) purification step (using a C18 SEP-Pack cartridge) was higher than 95%. This SPE final purification was nonetheless shown to dramatically reduce the final 68Ga-MAA outcome (>30% of the labelled MAA remain stuck on the SPE cartridge). All these published reports about the radio-labeling of MAA disclose the use of a crude fraction of the 68Ge/68Ga generator eluate directly. Because of the use of a crude fraction of the eluate, 68Ge breakthrough of the generator cannot be separated from the final product during the labeling procedure. Furthermore, this method uses only a part of the elutable 68Ga activity. To overcome this drawback, Mueller et al. (Mueller D., Kulkarni H., Baum R. P., Odparlik A., Rapid synthesis of 68Ga-labeled macroaggregated human serum albumin (MAA) for routine application in perfusion imaging using PET/CT. 2017; 122:72-77) recently provided a convenient preparation of 68Ga-MAA using a cationic prepurification of the generator eluate. The method allows the use of most of the eluted 68Ga activity from the generator and does not require any purification step of the reaction media as the 68Ge breakthrough is removed during the cationic prepurification. Nonetheless, in the absence of a final purification step, the production can be lost in case of a low-yield labelling. The authors also show that the MAA pre-washing step using centrifugation, to remove the tin chloride, is not necessary to achieve an efficient labelling yield.

Commercial Gallium-68 (68Ge/68Ga) generators are widely available. The parent isotope 68Ge has a half-life of 270.95 days and can be easily delivered to hospitals as a generator, where it can be used as the source of Ga-68 for at least 1 year. Short half-life 68Ga can be easily eluted from the generator at any time on the site of application. The 68Ga generator of a chromatographic type is a glass column with a sorbent based on modified TiO₂. The parent radionuclide 68Ge is fixed on this sorbent. The column is placed into a lead shielding container and provided with eluent and eluate lines. 68Ga, which is generated as a result of 68Ge decay, is eluted from the column for example using a 0.1 M HCl solution. The parent isotope activity is for example between 10mCi (370 MBq) and 100mCi (3700 MBq). 68Ge breakthrough is usually less than 0.005%.

99mTc-labeled-MAA is a widely used established lung perfusion agent using SPECT. Due to the superiority of PET over SPECT and the 99Mo upcoming shortage, 68Ga-labeled MAA for PET/CT perfusion imaging represents an attractive alternative to 99mTc-labeled MAA.

While the labeling conditions of 68Ga with MAA are well defined, there is a lack of an effective, easy to automatize on single use cassette-based system and of time/yield-efficient final purification of labelled 68Ga-MAA particles from the bulk reaction media, that would remove the 68Ge breakthrough, consistently assure a high radiochemical purity enabling safe patient injection and not impacting the final synthesis yield.

Nowadays, such final purification is performed either using centrifugation, which requires extra apparatus, is time consuming, negatively impacts the overall synthesis yield (˜20% activity loss) and is unwelcomed from a radioprotection point of view, or using SPE purification, which negatively impacts the yield (>30% activity loss due to labelled particles stuck on the cartridge). One recent example uses a cationic prepurification of the generator eluate, which is time consuming, without any final purification which is not filled from a regulatory point of view. Alternative synthesis methods with a very effective final purification step are thus highly desirable. The purification method of choice must be effective enough and reliable to ensure a high level of radiochemical purity.

### SUMMARY

In an embodiment, the present invention provides a method for synthesis and purification of radiolabeled macroaggregated human serum albumin (MAA) to form a bulk solution injectable to a patient, comprising: providing a radiometal in a generator, the radiometal comprising a generator eluate; synthesizing the radiolabeled MAA in a reactor with MAA particles from a commercially available labeling kit for 99mTc and the generator eluate so as to provide synthesized radiolabeled MAA particles; passing the synthesized radiolabeled MAA particles on a syringe filter membrane having a membrane composition, diameter, and pore size configured to trap the radiolabeled MAA particles as trapped radiolabeled MAA particles and not retain impurities from a bulk solution, the impurities comprising free radioactive metal isotopes, parent radioactive metal breakthrough, and stannous chloride present in the MAA labeling kit for 99mTc; and untrapping the trapped radiolabeled MAA particles from the syringe filter using a saline or buffered solution passing through the syringe filter in an opposite direction of a trapping movement as a final bulk solution, and providing the final bulk solution injectable to the patient into a vial.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 schematically represents radiometallabeled MAA trapping (left side, the particles are trapped on the filter, while the impurities pass through the filter to the waste) and untrapping (right side, the solution flow untraps the particles) steps.

### DETAILED DESCRIPTION

In an embodiment, the present invention provides a synthesis of 68Ga-labeled MAA particles, which is easily automated on ready-to-use consumables including an efficient final purification of the labelled particles.

In an embodiment, the present invention provides a method for the synthesis and purification of radiolabeled macroaggregated human serum albumin (MAA) to form a bulk solution injectable to a patient, wherein the method comprises the following steps of;
providing a radiometal in a generator, under the form of a generator eluate;
optionally prepurifying the generator eluate on a cationic cartridge and eluting the prepurified generator eluate;
synthesizing the radiolabeled MAA in a reactor with MAA particles from a commercially available labeling kit for 99mTc and said generator eluate, prepurified or not;
passing the synthesized radiolabeled MAA particles on a syringe filter membrane which membrane composition, diameter and pore size are chosen so as to trap the radiolabeled MAA particles, while impurities from the bulk solution are not retained, said impurities essentially consisting of free radiometal isotopes, parent radiometal breakthrough and stannous chloride present in said MAA labeling kit for 99mTc;
untrapping said trapped radiolabeled MAA particles from the syringe filter using a saline or buffered solution passing through the syringe filter in the opposite direction of the trapping movement and providing the final bulk solution injectable to a patient into a vial.

According to preferred embodiments, the method further comprises one of the following characteristics or a suitable combination thereof;
radiolabeled MAA particles to be purified are MAA particles labelled with detectable metal ions selected from the group of 99mTc, 94mTc, 48V, 52Fe, 55Co, 64Cu, 68Ga, 67Ga, 111In, 113In, 86Y, 89Zr, 203Pb, 212Bi, 82Rb, 186Re and 81mKr;
radiolabeled MAA particles to be purified are MAA particles labelled with detectable metal ions selected from the group of 99mTc, 68Ga, 86Y, 89Zr and 64Cu;
radiolabeled MAA particles to be purified are MAA particles labelled with 99mTc or 68Ga;
the syringe filter membrane pore size is in the range of 0.1-10.0 μm;
the syringe filter membrane pore size is in the range of 0.1-5.0 μm;
the syringe filter membrane pore size is in the range of 0.1-0.45 μm;
the syringe filter membrane diameter is in the range of 10-33 mm;
the syringe filter membrane diameter is in the range of 20-33 mm;
the syringe filter membrane is a low-protein binding hydrophilic membrane selected from the group consisting of PVDF, PES, CA, hydrophilic PTFE, nylon, Glass Fiber, RC, CE, CN and PP;
the syringe filter is a single-use filter cartridge possibly having Luer lock fittings;
the syringe filter is attached to a single-use cassette in an automated process;
the generator eluate is maintained at room temperature during 2 to 30 minutes or heated at 40-80° C. during 2 to 20 minutes before the step of trapping the generator eluate on the syringe filter;
the reactor for synthesizing the radiometallalabeled MAA is an automated synthesizer.

The method of the present invention allows the purification of 68Ga-labeled MAA particles, prepared using directly the whole generator eluate or alternately including a cationic prepurification of the generator eluate. The method is furthermore compatible to any commercially available MAA labelling kits for 99mTc.

This efficient purification is made by the use of a syringe filter. A syringe filter is a single-use filter cartridge. Syringe filters may have Luer lock fittings, though not universally so. For manual purification, it is attached to the end of a syringe for use. For automated processes, syringe filters can be fixed on single use cassettes. The use of a needle is optional; where desired it may be fitted to the end of the syringe filter. A syringe filter generally consists of a plastic housing with a membrane which serves as a filter. The fluid to be purified may be cleaned by drawing it up through the filter. A syringe filter membrane is characterized by its composition (material and pore size) and its diameter. Common pore sizes available are 0.1, 0.2, 0.22, 0.45, 5 and 10 μm, although intermediate pore sizes are easily available. Membrane diameters of 10, 13, 25, 33 mm are common as well. The syringe filter body may be made of materials such as polypropylene and nylon. The filter membrane may be of polytetrafluoroethylene (PTFE), nylon, cellulose acetate (CA), polyvinylidene fluoride (PVDF), cellulose ester (CE), polyethersulfone (PES), polypropylene (PP), Glass Fiber (GF), regenerated cellulose (RC), cellulose nitrate (CN), etc.

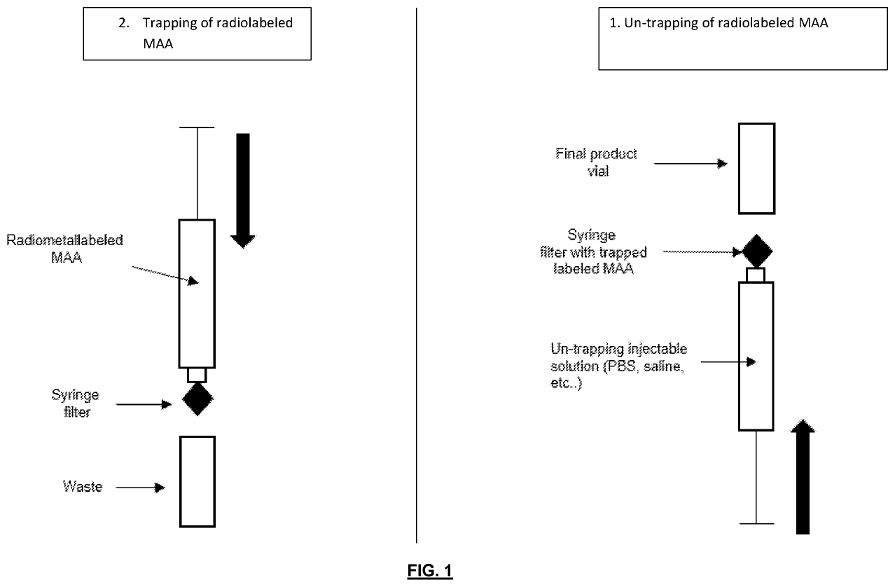

While passing through the reaction media on the syringe filter membrane, the labelled and unlabelled MAA particles are retained on the filter due to size exclusion, while the 68Ge breakthrough and remaining free 68Ga3+ pass through the syringe filter membrane to the waste (FIG. 1, left).

To untrap the particles from the syringe filter, a solution is passed through the syringe filter membrane, in the opposite (or reverse) direction of the trapping movement, to a final product vial (FIG. 1, right). The untrapping is due to the flow of the untrapping solution. The aforementioned untrapping solution being injectable (e.g. an appropriate phosphate buffer solution or physiological saline, i.e. 0.154 mol/L or 9 g/L NaCl), the resulting tracer solution is readily injectable to a patient.

It brings several advantages: a reduction of the preparation duration, which results in an increase of the overall yield; a simplification of the automated equipment needed for the synthesis of the radiopharmaceutical; a purification process which is compatible with any radiolabeled MAA particles, thus which is not limited to 68Ga or Tc99m; the assurance of a high level radiochemical purity even in case of a low-yield labelling.

According to the present invention, the purification process is performed by passing the bulk of the synthesis of the radiolabeled MAA particles on a syringe filter, that may be placed on a single-use cassette for automation. This syringe filter has the characteristic to retain the labelled (and unlabelled) MAA products but not the free unlabelled radioisotope (i.e. 68Ga3+ in the case of 68Ga-MAA labelling, also the 68Ge breakthrough) assuring a high level of radiochemical purity, and not the tin chloride coming from the original MAA labelling kit either.

In some embodiments of the present invention, the radiolabeled MAA particles to be purified are MAA particles labelled with a detectable metal ion such as 99mTc, 94mTc, 48V, 52Fe, 55Co, 64Cu, 68Ga, 67Ga, 111In, 113In, 86Y, 89Zr, 203Pb, 212Bi, 82Rb, 186Re, 81mKr.

In some preferred embodiments of the present invention, the radiolabeled MAA particles to be purified are MAA particles labelled with 99mTc, 68Ga, 86Y, 89Zr or 64Cu.

In some preferred embodiments of the present invention, the radiolabeled MAA particles to be purified are MAA particles labelled with 99mTc or 68Ga.

In some embodiments, the syringe filter membrane pore size is in the range of 0.1-10.0 μm.

In some preferred embodiments, the syringe filter membrane pore size is in the range of 0.1-5.0 μm.

In some preferred embodiments, the syringe filter membrane pore size is in the range of 0.1-0.45 μm.

In some embodiments, the syringe filter membrane diameter is in the range of 10-33 mm.

In some embodiments, the syringe filter membrane diameter is in the range of 20-33 mm.

In some embodiments, the syringe filter membrane is selected from the group of low-protein binding hydrophilic membranes (PVDF, PES, CA, hydrophilic PTFE, nylon, Glass Fiber, RC, CE, CN, PP).

### EXAMPLES

### Example 1

This example shows the efficiency of using a syringe filter to purify a bulk of 68Ga-MAA. The 68Ga-MAA was synthesized on an automated synthesizer, using a cationic prepurification of the generator eluate: an Eckert & Ziegler 68Ge/68Ga generator is eluted with 5 mL 0.1 M HCl. The generator eluate is trapped on a PS-H+ cationic cartridge that retains the eluted 68Ga3+. The activity is then eluted to the reactor using an acidified concentrated NaCl solution. The MAA from a Pulmocis® labeling kit, dissolved in an acetate buffer, is added to the reactor. After a heating time of 6 minutes at 60° C., the reaction media is sent to the final product vial and formulated with a phosphate buffer to yield a final pH of 7.0 (final volume is 10 mL). Non-decay corrected (n.d.c.) radiochemical yield is of 75% (111.4 MBq) and the radiochemical purity is of 80%. This final product solution (111.4 MBq) was manually passed through a 25 mm, 5 μm pore size PVDF syringe filter membrane (Millipore Ref. SLSV025LS). The whole labeled 68Ga-MAA particles are retained on the filter (activity on the filter: 88.6 MBq) while the free 68Ga3+ passes through the filter (activity in the filtrate: 22.8 MBq). After trapping, 10 mL of physiological saline are passed through the syringe filter, in the opposite direction of the trapping movement, to untrap the labeled particles. An efficient untrapping of 98.2% (2 MBq remaing on the filter) is reached. A thin layer chromatography (TLC) analysis showed a radiochemical purity of the untrapped 68Ga-MAA particles of 98.9%.

### Example 2

This example shows the efficiency of using a syringe filter to trap and untrap a bulk of 99mTc-MAA. The 99mTc-MAA is synthesized using a commercial Pulmocis® labeling kit following the routine procedure: the Tc-generator is directly eluted into the Pulmocis⁻ labeling kit. After 15 minutes at room temperature under a gentle mixing, the 99mTc-labeled MAA bulk solution was manually passed through a syringe filter (25 mm diameter, 5 μm pore size, PVDF membrane, Millipore Ref. SLSV025LS). The labelled particles are retained on the filter (activity on the filter: 44163 cps/10 s). After trapping, 10 mL physiological saline passes through the filter in the opposite direction of the trapping movement. The untrapping efficiency is of 91% (activity remaining on the filter: 4523 cps/10 s).

### Examples 3-11

The following examples show the efficiency of using a syringe filter to purify a bulk of 68Ga-MAA. The 68Ga-MAA is synthesized on an automated synthesizer, with no prepurification of the generator eluate (i.e. using the whole generator eluate), but with a final purification on a syringe filter placed on the single-use cassette. An Eckert & Ziegler 68Ge/68Ga generator is eluted with 5 mL 0.1 M HCl directly to the reactor that contains the MAA particles from a Pulmocis® labeling kit dissolved in 2 mL of a 0.35 M acetate solution. After a heating time of 6 minutes at 60° C., the reaction media is passed through the syringe filter that retains the labeled particles, while the free 68Ga3+ and the 68Ge breakthrough pass through the filter to the waste. 10 mL physiological saline are then used to untrap the labelled particles to the final product vial. Synthesis time is of 12 minutes after generator elution. Experiments were repeated varying the filter syringe type (Examples 3-11). Table 1 show the non-decay corrected (n. d. c.) radiochemical yield (RCY), the radiochemical purity (RCP) and the 68Ge content (when measured) in the final product vial for Examples 3-11.

| | | RCY | RCP (%) | 68Ge |
|---|---|---|---|---|
| Ex. No | Filter | (% n.d.c.) | at t0 | content(*) |
| 3 | 25 mm, 5 μm, PVDF | 46 | 94.5 | / |
| | Millex Ref. SLSV025LS | | | |
| 4 | 13 mm, 5 μm, CA | 46 | 99.1 | / |
| | Steriltech Ref. CA501350 | | | |
| 5 | 13 mm, 0.45 μm, PVDF | 19 | 98.7 | / |
| | Millex Ref. SLHVX13TL | | | |
| 6 | 25 mm, 0.22 μm, PVDF | 80 | 100 | <0.0001% |
| | Millex Ref. SLGV0250S | | | |
| 7 | 25 mm, 0.22 μm, PVDF | 82 | 100 | <0.0001% |
| | Millex Ref. SLGV0250S | | | |
| 8 | 25 mm, 0.22 μm, PVDF | 83 | 100 | / |
| | Millex Ref. SLGV0250S | | | |
| 9 | 25 mm, 0.22 μm, PVDF | 81 | 100 | / |
| | Millex Ref. SLGV0250S | | | |
| 10 | 25 mm, 0.22 μm, PVDF | 83 | 99.5 | / |
| | Millex Ref. SLGV0250S | | | |
| 11 | 25 mm, 0.22 μm, PVDF | 84 | 99.7 | / |
| | Millex Ref. SLGV0250S | | | |

(*)Measured after complete decay of 68Ga3+. The limit for patient injection is of 0.001% of the initial activity.

### Table 1—Results of Examples 3-11

Table 1 shows the high efficiency of using syringe filters for the purification of radiolabeled MAA. High-level radiochemical yield is almost always achieved, even in cases of low labeling yield and of low untrapping yield. The procedure is also time-effective, as the synthesis time, including the final purification and dispensing to the final product vial, is of 12 minutes after the generator elution.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for synthesis and purification of radiolabeled macroaggregated human serum albumin (MAA) to form a bulk solution injectable to a patient, comprising:
providing a radiometal in a generator, the radiometal comprising a generator eluate;
synthesizing the radiolabeled MAA in a reactor with MAA particles from a commercially available labeling kit for 99mTc and the generator eluate so as to provide synthesized radiolabeled MAA particles;
passing the synthesized radiolabeled MAA particles on a syringe filter membrane having a membrane composition, diameter, and pore size configured to trap the radiolabeled MAA particles as trapped radiolabeled MAA particles and not retain impurities from a bulk solution, the impurities comprising free radioactive metal isotopes, parent radioactive metal breakthrough, and stannous chloride present in the MAA labeling kit for 99mTc; and
untrapping the trapped radiolabeled MAA particles from the syringe filter using a saline or buffered solution passing through the syringe filter in an opposite direction of a trapping movement as a final bulk solution, and providing the final bulk solution injectable to the patient into a vial.

2. The method of claim 1, wherein radiolabeled MAA particles to be purified comprise MAA particles labelled with detectable metal ions comprising at least one of 99mTc, 94mTc, 48V, 52Fe, 55Co, 64Cu, 68Ga, 67Ga, 111In, 113In, 86Y, 89Zr, 203Pb, 212Bi, 82Rb, 186Re, or 81mKr.

3. The method of claim 1, wherein radiolabeled MAA particles to be purified comprise MAA particles labelled with detectable metal ions comprising at least one of 99mTc, 68Ga, 86Y, 89Zr, or 64Cu.

4. The method of claim 1, wherein radiolabeled MAA particles to be purified comprise MAA particles labelled with 99mTc or 68Ga.

5. The method of claim 1, wherein the pore size is in a range of 0.1-10.0 μm.

6. The method of claim 5, wherein the pore size is in a range of 0.1-5.0 μm.

7. The method of claim 6, wherein the pore size is in a range of 0.1-0.45 μm.

8. The method of claim 5, wherein the diameter is in a range of 10-33 mm.

9. The method of claim 8, wherein the diameter is in a range of 20-33 mm.

10. The method of claim 8, wherein the syringe filter membrane comprises a low-protein binding hydrophilic membrane comprising at least one of PVDF, PES, CA, hydrophilic PTFE, nylon, Glass Fiber, RC, CE, CN, or PP.

11. The method of claim 1, wherein the syringe filter comprises a single-use filter cartridge.

12. The method of claim 1, wherein the syringe filter is attached to a single-use cassette in an automated process.

13. The method of claim 1, wherein the generator eluate is maintained at room temperature for 2 to 30 minutes or heated at 40-80° C. for 2 to 20 minutes before trapping the generator eluate on the syringe filter.

14. The method of claim 1, wherein the reactor for synthesizing the radiolabeled MAA comprises an automated synthesizer.

15. The method of claim 1, further comprising:
prepurifying the generator eluate on a cationic cartridge as a prepurified generator eluate, and eluting the prepurified generator eluate.

16. The method of claim 11, wherein the single-use filter cartridge comprises Luer lock fittings.
